(19) United States
(12) Patent Application Publication
(10) Pub. No.: US 2022/0151986 A1
(43) Pub. Date: May 19, 2022

(54) MDMA PRODRUGS TO ASSIST PSYCHOTHERAPY

(71) Applicant: Mind Medicine, Inc., New York, NY (US)

(72) Inventors: Matthias Emanuel LIECHTI, Oberwil (CH); Felix LUSTENBERGER, Cham (CH); Daniel TRACHSEL, Detligen (CH)

(73) Assignee: Mind Medicine, Inc., New York, NY (US)

(21) Appl. No.: 17/518,846

(22) Filed: Nov. 4, 2021

Related U.S. Application Data
(60) Provisional application No. 63/115,245, filed on Nov. 18, 2020.

(51) Int. Cl. A61K 31/36

(52) U.S. Cl. CPC A61K 31/36

(57) ABSTRACT

A compound including a prodrug having a psychoactive base substance attached to an amino acid. A method of treating an individual, especially in substance-assisted psychotherapy, by administering proMDMA or a proMDMA-like compound to the individual, metabolizing the prodrug, and releasing the MDMA or MDMA-like substance in the individual. A method of reducing anxiety while administering MDMA, by providing a slow release of MDMA or an MDMA-like substance and thereby reducing anxiety in the individual at the onset of administration. A method of personalized medicine, by evaluating an individual and determining if there are characteristics of the individual present that would not be suitable for MDMA treatment and administering proMDMA or a proMDMA-like substance to the individual. A method of reducing abuse of MDMA, by administering proMDMA or a proMDMA-like substance, and providing a delayed and attenuated effect of MDMA or a MDMA-like substance, thereby reducing abuse.

### BACKGROUND OF THE INVENTION

### 1. Field of the Invention

The present invention relates to novel substances (compositions of matter) for substance-assisted psychotherapy including (1) the description of new substances, (2) methods of synthesis of the substances, and (3) applications of the substances in treating medical conditions.

### 2. Background Art

3,4-Methylenedioxymethamphetamine (MDMA) is a psychoactive drug that alters mood and perception, and is investigated as an adjunct in psychotherapy for posttraumatic stress disorder (PTSD), social anxiety, autism (Danforth, 2016; Danforth et al., 2018; Danforth et al., 2016; Mithoefer et al., 2019; Mithoefer et al., 2010; Oehen et al., 2013), and may later also be studied and used for a range of other medical conditions. Such conditions where MDMA or related substances may be useful include, but is not limited to, substance-use disorder, depression, anxiety disorder, anxiety with life-threatening disease, personality disorder including narcistic and antisocial disorder, and obsessive-compulsive disorder. MDMA or related substances can also be used to enhance couple therapy.

MDMA and related substances are thought to produce positive therapeutic long-term effects in the context of MDMA/substance-assisted psychotherapy by producing acute subjective positive mood effects that also enhance the effectiveness of psychotherapy and can be beneficial on their own. Such acute beneficial MDMA-effects include, but are not limited to, feelings of well-being, feelings of connectivity to others, feelings of increased trust, feelings of love, enhanced emotional empathy, and enhanced feelings of pro-sociality and prosocial behavior (Hysek et al., 2014; Liechti et al., 2001; Schmid et al., 2014; Vollenweider et al., 1998a).

Prior art discloses the use of substances in substance-assisted psychotherapy including MDMA, psilocybin, and LSD (Carhart-Harris et al., 2017; Liechti, 2017; Luoma et al., 2020; Nichols et al., 2017; Sessa et al., 2019; Trope et al., 2019). However, other substances may be more suitable with different therapeutic benefits/tolerability profiles. Additionally, MDMA is the only empathogen-type substance currently investigated for substance-assisted psychotherapy while psilocybin and LSD are psychedelics with a different effect profile and mode of action (Holze et al., 2020). Alternatives to MDMA have been suggested (Oeri, 2020). These alternative MDMA-like substances include many compounds that may share some similarity with MDMA based on their in vitro pharmacological profiles and based on reports of their subjective effects by recreational users (Oeri, 2020). 3,4-Methylenedioxamphetamine (MDA) is the only MDMA-like substance which has been used to assist psychotherapy in the past (Baggott et al., 2019; Yensen et al., 1976).

The present invention includes an alternative approach to optimize effects of MDMA and MDA by using a pro-drug approach. This allows modification of the MDMA and MDA effects but at the same time the novel compounds used will be transformed to the known and previously used active substances MDMA and MDA in the body providing higher safety compared to a compound with a novel structure of the active entity. MDMA may not be the only compounds suitable for substance-assisted therapy. In fact, MDMA may be contraindicated in some subjects (for example due to cardiovascular side effects) and substance characteristics slightly different from those of MDMA may be needed in some patients.

Substances with expected overall similar benefits as those of MDMA in MDMA-assisted therapy are needed, while such novel substances could be improved regarding some of the adverse effects of MDMA or may exhibit properties in addition to MDMA that are of therapeutic interest. Therefore, the present invention describes novel MDMA-like compounds that could substitute for MDMA in selected patients.

Substances with overall MDMA-like properties are those with an overall similar in vitro pharmacological profile and namely substances which release monoamines with a preference for release of serotonin (5-HT) over dopamine (DA) (Liechti, 2014; Oeri, 2020; Simmler et al., 2013).

While MDMA acutely induces mostly positive subjective effects including heightened mood, openness, trust, and enhanced empathy, there can also be negative drug effects including anxiety in particular at the onset of the subjective response (Hysek et al., 2014; Liechti et al., 2001; Schmid et al., 2014; Vollenweider et al., 1998a).

A possible solution to mitigate anxiety at onset consists of slowing the onset of the drug effect by using a slow-release formulation of MDMA. The present invention newly uses a prodrug that is expected to be slowly converted to MDMA or a MDMA-like substance in the body and thereby producing a slower and attenuated response with reduced anxiety at onset of the subjective drug effect.

Amphetamines including MDMA carry a risk of abuse liability. This is evidenced by the fact that MDMA is self-administered by animals, although not very robustly (Cole & Sumnall, 2003; Creehan et al., 2015), promotes conditioned place preference (Cole & Sumnall, 2003) and releases dopamine (Kehr et al., 2011) in the brain similar to, although not as robustly, as other drugs of abuse. The risk of abuse of a substance with central-nervous system action is generally associated in part with the rapidity of the onset of the subjective drug effect, which is linked to the rapidity of the drug-plasma concentration increase in the brain (or blood plasma) (Busto & Sellers, 1986; Mumford et al., 1995).

One way of reducing the addictive property of a substance of abuse is by slowing the onset of action and/or the increase in the blood concentration, for example, by using slow-release formulations (Mumford et al., 1995).

Another approach is to use a prodrug that is slowly converted to the active substance. For example, this approach has been used with the prodrug lisdexamfetamine, which is converted to d-amphetamine after reaching the circulation (Jasinski & Krishnan, 2009a; Jasinski & Krishnan, 2009b).

Therefore, there remains a need for methods of administering MDMA to individuals safely and minimizing unwanted side effects.

### SUMMARY OF THE INVENTION

The present invention provides for a compound including a prodrug having a psychoactive base substance attached to an amino acid.

The present invention provides for a method of treating an individual, especially in substance-assisted psychotherapy, by administering proMDMA or a proMDMA-like compound to the individual, metabolizing the prodrug, and releasing the MDMA or MDMA-like substance in the individual.

The present invention also provides for a method of reducing anxiety while administering MDMA, by providing a slow release of MDMA or an MDMA-like substance and thereby reducing anxiety in the individual at the onset of administration.

The present invention provides for a method of personalized medicine, by evaluating an individual who is in need of MDMA treatment and determining if there are characteristics of the individual present that would not be suitable for MDMA treatment and administering proMDMA or a proMDMA-like substance to the individual.

The present invention provides for a method of reducing abuse of MDMA, by administering proMDMA or a proMDMA-like substance, and providing a delayed and attenuated effect of MDMA or a MDMA-like substance, thereby reducing abuse.

### DESCRIPTION OF THE DRAWINGS

Other advantages of the present invention are readily appreciated as the same becomes better understood by reference to the following detailed description when considered in connection with the accompanying drawings wherein:

FIGS. 1A-1H show examples of MDMA-like substances. 3,4-methylenedioxymethamphetamine (MDMA) (1A), 3,4-methylenedioxyamphetamine (MDA) (1B), 1-(1,3-benzodioxol-5-yl)-methyl-2-butanamine (MBDB) (1C), 3,4-methylenedioxyethylamphetamine (MDEA) (1D), methylone (1E), 5-(2-aminopropyl)-benzofuran (5-APB) (1F), N-methyl-1-(benzofuran-5-yl)-propane-2-amine (5-MAPB) (1G), 5,6-methylenedioxy-2-aminoindane (MDAI) (1H);

FIG. 2 shows lysMDA and lysMDMA as representative examples of proMDMA or proMDMA-like compound structures, inactive lysMDA or lysMDMA is rapidly absorbed after oral administration in the intestine as shown for related compounds (Hutson et al., 2014), and peptidases in the blood metabolize lysMDA or lysMDMA to lysine and active MDA or MDMA, respectively;

FIG. 3 is a graph showing the plasma alprazolam levels after administration of immediate-release (IR) and extended-release (XR) formulation;

FIG. 4 is a graph showing subjective effects of immediate-release (IR) and extended-release (XR) formulations of alprazolam on the subjective effect-time curves (Mumford et al., 1995);

FIGS. 5A-5B are graphs showing the effect of immediate-release and extended-release formulations of alprazolam on maximal drug-liking ratings (FIG. 5A) and associated drug-reinforcement measures (FIG. 5B) (Mumford et al., 1995);

FIG. 6A is a graph showing plasma levels of d-amphetamine after administration of the prodrug lisdexamfetamine and d-amphetamine at equivalent molar doses (Jasinski et al., 2009b) in humans, the drugs were administered intravenously, and FIG. 6B is an inset showing detail from 0 to 1 hour;

FIG. 7A is a graph showing subjective drug-liking ratings as a measure of abuse liability after administration of the prodrug lisdexamfetamine and d-amphetamine at equivalent molar doses (Jasinski et al., 2009b), the drugs were administered intravenously, and FIG. 7B is an inset showing detail from 0 to 1 hour;

FIG. 8 is a graph showing subjective peak changes after administration of the prodrug lisdexamfetamine at doses of 50 mg, 100 mg, and 150 mg and a 100 mg equivalent dose of d-amphetamine (40 mg) orally;

FIG. 9 is a graph showing systolic blood-pressure values after administration of the prodrug lisdexamfetamine at doses of 50 mg, 100 mg, and 150 mg and a 100 mg equivalent dose of d-amphetamine (40 mg) orally;

FIG. 10A is a graph (semilog plot as inset shown in FIG. 10B) of the plasma concentrations of amphetamine after administration of lisdexamfetamine and d-amphetamine at equivalent doses;

FIG. 11 is a graph of the subjective liking-rating scores over time after administration of lisdexamfetamine and amphetamine to healthy subjects;

FIG. 12 is a graph of the systolic blood pressure over time after administration of lisdexamfetamine and amphetamine to healthy subjects; and

FIG. 13 is a graph of the acute effects of MDMA and amphetamine illustrating higher and shorter MDMA effects on drug liking compared with amphetamine and indicating room for attenuating the MDMA effect using a prodrug concept.

### DETAILED DESCRIPTION OF THE INVENTION

The present invention generally provides for novel MDMA-like compounds, descriptions of their production and of their use, and use advantages over existing substances used in substance (MDMA)-assisted psychotherapy to treat medical conditions. Most generally, the present invention provides for a compound of a prodrug including a psychoactive base substance attached to an amino acid. Preferably, the compounds are prodrugs of MDMA and MDMA-like compounds.

A “prodrug” as used herein, refers to a compound that includes a moiety attached to an active drug substance that is metabolized after administration to an individual and the compound is converted into the active drug substance. Using a prodrug allows for improving how the active drug is absorbed, distributed, metabolized, and excreted. Prodrugs can be used to prevent release of the active drug in the gastrointestinal tract upon administration so that the drug can be released more favorably elsewhere in the body. The prodrugs in the present invention can be referred to as “proMDMA” or “proMDMA-like compound”.

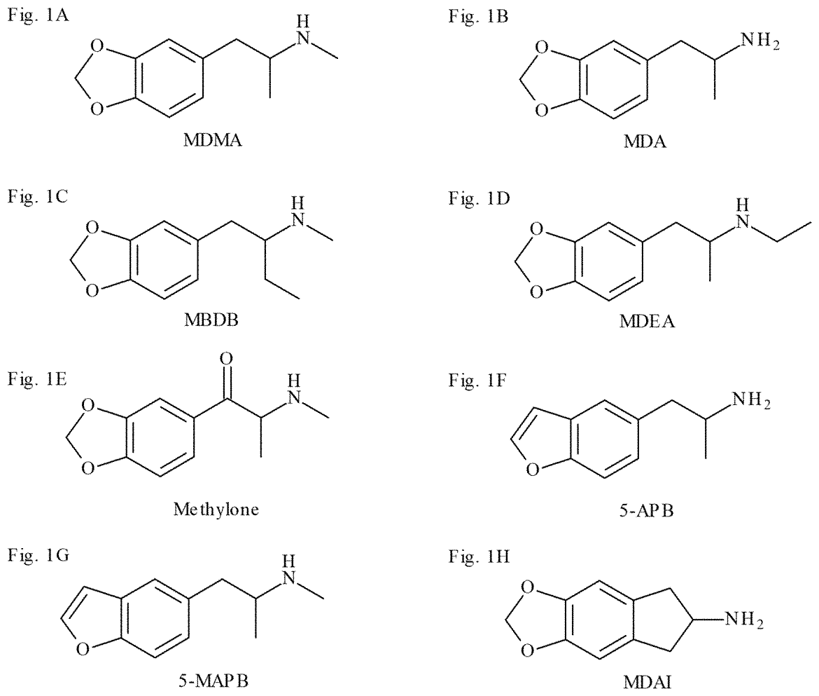

More specifically, the compound includes an amino acid covalently attached to a psychoactive base substance of MDMA or an MDMA-like compound (FIGS. 1A-1H). The addition of the amino acid makes the active compound inactive mainly by preventing interaction with monoamine transporter, which is the site of action but also affecting bioavailability/rate of absorption. The amino acid can be lysine or any other amino acid such as alanine, arginine, asparagine, aspartic acid, cysteine, glutamine, glutamic acid, glycine, histidine, isoleucine, leucine, methionine, phenylalanine, proline, serine, threonine, tryptophan, tyrosine, or valine and typically attached to the amine (N)-group of MDMA or the MDMA-like substance and hence reducing pharmacological activity at the primary site of action (cell-membrane monoamine transporters including serotonin, dopamine and norepinephrine transporter), and also altering extent and rate of absorption and mainly releasing active substance in the circulation after absorption of the inactive compound. The amino acid can be any other natural or synthetic amino acid. The invention will be described with lysine as amino acid example combined with MDMA and MDA. However, the invention can use any other amino acid covalently bound to any other MDMA-like substance via the amine group of the MDMA-like substance to form a peptide bond.

The MDMA-like compound can be MDMA (FIG. 1A), 3,4-methylenedioxyamphetamine (MDA) (FIG. 1B), 3,4-methylenedioxyethylamphetamine (MDEA) (FIG. 1D), 1-(1,3-benzodioxol-5-yl)methyl-2-butanamine (MBDB) (FIG. 1C), 1-(1,3-benzodioxol-5-yl)-2-aminobutane (BDB, also known as MDB) methylone (FIG. 1E), ethylone, 5,6-methylenedioxy-2-aminoindane (MDAI) (FIG. 1H), 5-iodo-2-aminoindane (5-IAI), 4-(2-aminopropyl)-benzofuran (4-APB), 5-(2-aminopropyl)-benzofuran (5-APB) (FIG. 1F), 6-(2-aminopropyl)-benzofuran (6-APB), N-methyl-1-(2,3-dihydrobenzofuran-5-yl)-propan-2-amine (5-MAPDB), 6-(2-methylaminopropyl)-benzofuran (6-MAPB) (FIG. 1G), or other compounds, namely a benzofuran, aminoindane or cathinone or mixed dopaminergic-serotonergic amphetamine and their N-alkylated analogs, with an MDMA-like pharmacological profile (Rickli et al., 2015a; Rickli et al., 2015b; Simmler et al., 2013) or active metabolites of such substances (Luethi et al., 2019). There is similarity of the structures in FIGS. 1A-1H, all of the compounds contain a 3,4-substitution of the benzene ring in the phenethylamine structure which is typical for MDMA-like compounds that preferably act on serotonin versus dopamine transporters to primarily release serotonin. Compounds can be used in any suitable pharmaceutical salt form such as hydrochloride or dimesylate, etc. Any active metabolites can also be used.

The invention described herein describes in detail two examples of substances representing the invention regarding substance matters including lysMDMA (for lysine covalently bound to MDMA) and lysMDA (for lysine covalently bound to MDA).

Compounds in the field of the present invention can generally be prepared in analogy to known routes such as described for lisdexamfetamine (patent numbers: WO2005032474A2, WO2006121552A2, U.S. Pat. No. 7,223,735B2, US2009234002A1, US20120157706A1, WO2017098533A2) which is derived from the combination of lysine as amino acid and dexamphetamine as psychoactive substance. Briefly, bis-N-protected lysine or another amino acid is activated at the carboxyl group by introducing a leaving group such as O-succinimide. In the present example, this activated lysine derivative is then allowed to react with a primary or secondary amine such as MDA or MDMA, respectively, to form the corresponding amide in the presence of a suitable non-protic base such as triethylamine, N-methylmorpholine or diisopropylethylamine. Tetrahydrofuran (THF) or dioxane is used as a suitable solvent, but others such as dimethylformamide (DMF) or dimethylsulfoxide (DMSO) may also be considered. After isolation and purification, the compounds such as bis-N-protected lysMDA or lysMDMA are redissolved in a suitable solvent and treated with the corresponding conditions to allow deprotection, e.g., the use of an acid to remove tert-butoxycarbonyl (BOC) groups or hydrogen in the presence of a catalyst such as palladium on activated charcoal (Pd—C) to remove hydrogen-sensitive protecting groups. The final products can either be isolated as a salt from corresponding conditions or as their free base. An optional further purification step and/or conversion to a salt such as hydrochlorides or mesylates by known procedures will lead to the final products such as lysMDA or lysMDMA or any similar combination of an MDMA-like psychoactive substance linked with an amino acid.

A problem relating to using MDMA in the treatment of medical conditions is that MDMA has some abuse liability due to its amphetamine structure and pharmacology. Namely, MDMA releases dopamine (Kehr et al., 2011), which is associated with dependence. MDMA also releases serotonin (Kehr et al., 2011), which counteracts dependence (Suyama et al., 2016). Due to its combined dopaminergic and serotonergic properties, MDMA is considered a moderate reinforcer compared to methylphenidate, cocaine or nicotine, which are strong reinforcers (Liechti, 2014). Nevertheless, abuse of MDMA can be a medical concern.

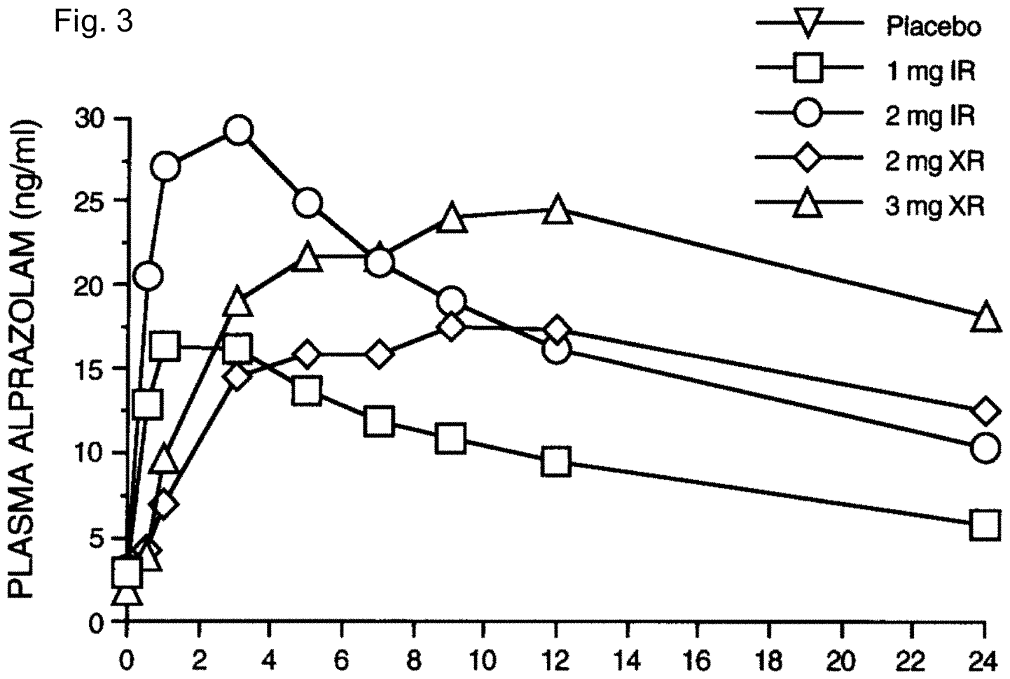

A measure of abuse liability that can easily be measured is subjective drug liking (Jasinski, 2000; Jasinski & Krishnan, 2009a; Jasinski & Krishnan, 2009b). Subjective effects of drug liking are thought to be associated with abuse liability. In particular, higher drug-liking scores and more rapidly increasing scores after substance administration are predictors of greater abuse liability. Consistently, immediate release formulations increase liking more rapidly and to higher levels than extended releaser formulations of a given central-nervous-system-acting substance. For example, this has been shown for alprazolam immediate-release and extended-release formulations with the extended-release formulation producing lower liking and less drug reinforcement compared to the rapid-release formulation (FIGS. 3-5B) (Mumford et al., 1995).

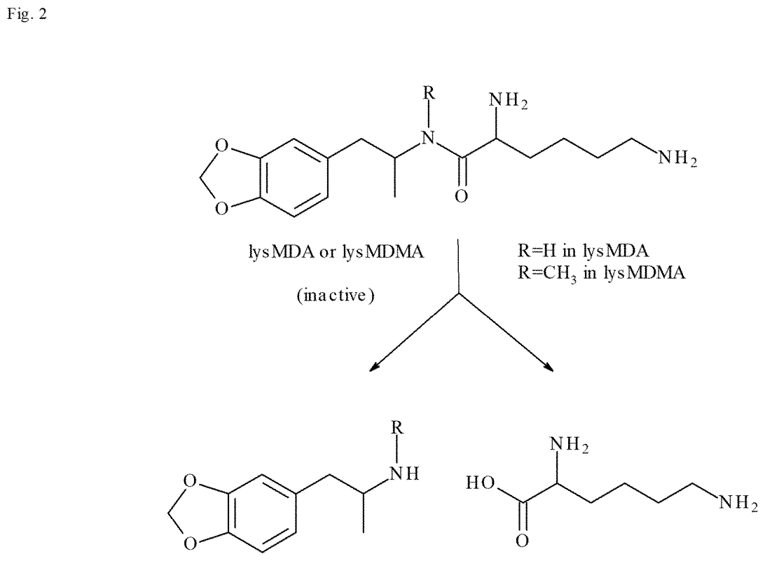

As illustrated for example in FIG. 2, the proMDMA-like compound is inactive and absorbed well after oral administration in the intestine where it is transported into the blood. In the blood, the proMDMA-like compound is cleaved into an amino acid (lysine in the example) and the active MDMA-like compound (MDA in the example in FIG. 2) as shown for related compounds (Hutson et al., 2014).

The cleaved amino acids are physiologically available and metabolically needed substances (protein synthesis) that are used by the body physiologically or metabolized as in the case of amino acids administered within food (meat) or food supplements.

The amino acid tryptophan can also be used and can be particularly useful in the present invention because it is the precursor amino acid used by the brain to produce the neurotransmitter serotonin (5-hydroxytryptamine, 5-HT). MDMA and MDMA-like substances release endogenous serotonin and can lead to serotonin depletion which in turn can lead to depressed mood a few days after MDMA administration. The tryptophan contained in tryptophan-MDMA prodrug helps prevent such serotonin depletion and associated negative mood effects.

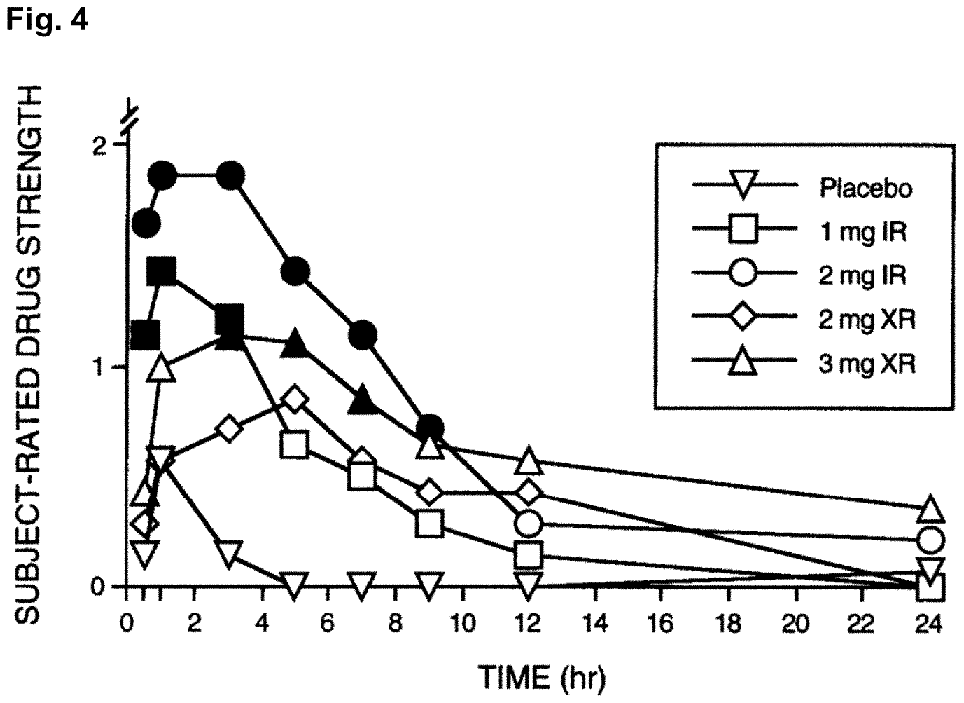

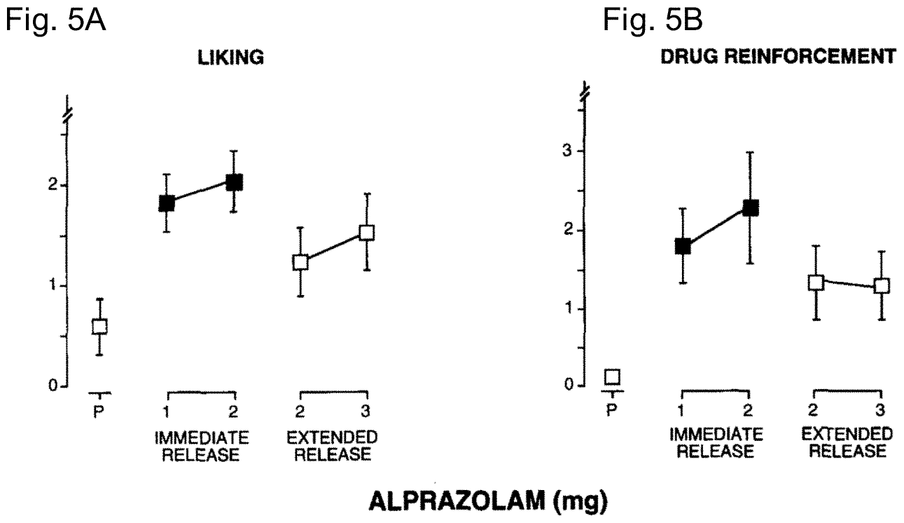

ProMDMA compounds have a low bioavailability when used via parenteral routes such as intranasal (snorting) or intravenous administration, limiting their abuse liability as shown for related compounds (FIGS. 4 and 5A-5B). This concept has previously been employed for d-amphetamine (U.S. Pat. No. 7,655,630B2) (Jasinski et al., 2009b) but not with MDMA or its analogs.

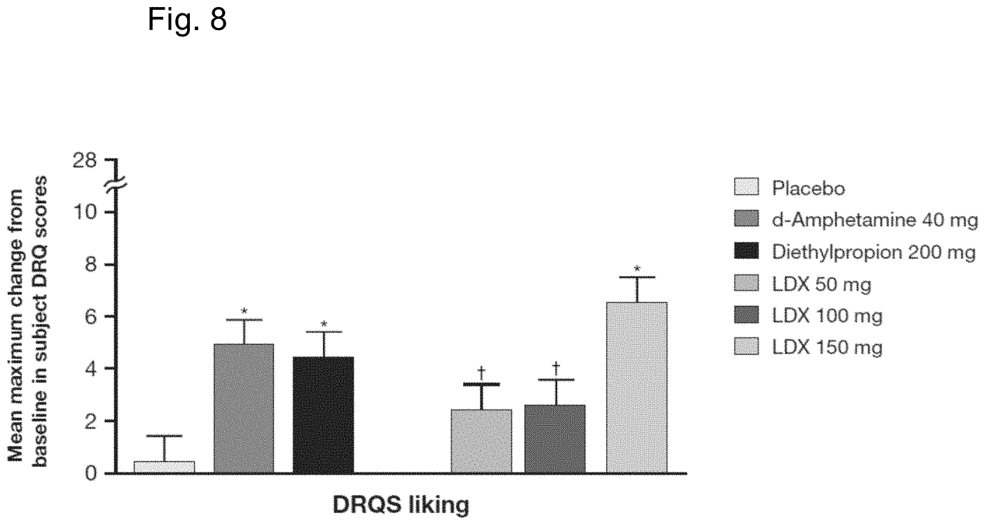

ProMDMA compounds can induce lower drug-liking ratings compared to equivalent doses of the mother substance. This has been shown using lisdexamfetamine and an equivalent oral dose of d-amphetamine (Jasinski et al., 2009a) (FIG. 8) and can be confirmed using lysMDMA/lysMDA and MDMA/MDA in the clinical studies used to further support the present invention. In FIG. 8, ratings of liking for lisdexamfetamine were lower compared to d-amphetamine rating scores.

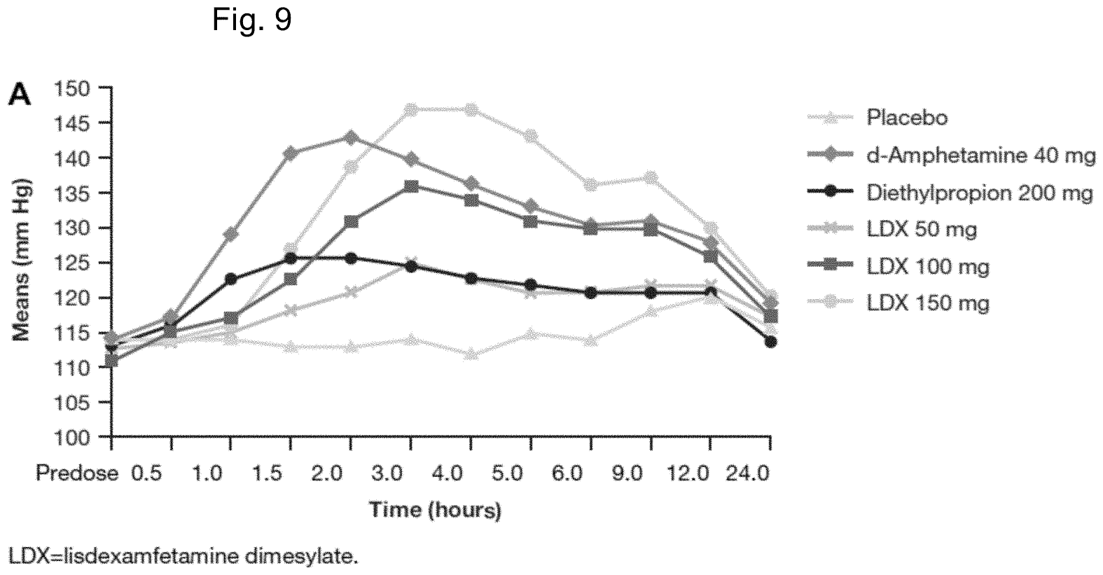

MDMA and related substances increase blood pressure rapidly and, in some subjects, markedly (Hysek et al., 2011; Vizeli & Liechti, 2017). This can be a problem for subjects or patients with cardiovascular disease. MDMA-like substances with lower acute cardiovascular effects or an attenuated increase in blood pressure are warranted. ProMDMA and proMDMA-like compounds exhibit an attenuated cardio-stimulant response due to the slowed production of the active substance from the prodrug as similarly shown for lisdexamfetamine and d-amphetamine (Jasinski et al., 2009a) (FIG. 9). In FIG. 9, blood pressure after 100 mg lisdexamfetamine increased more slowly and later compared to administration of d-amphetamine.

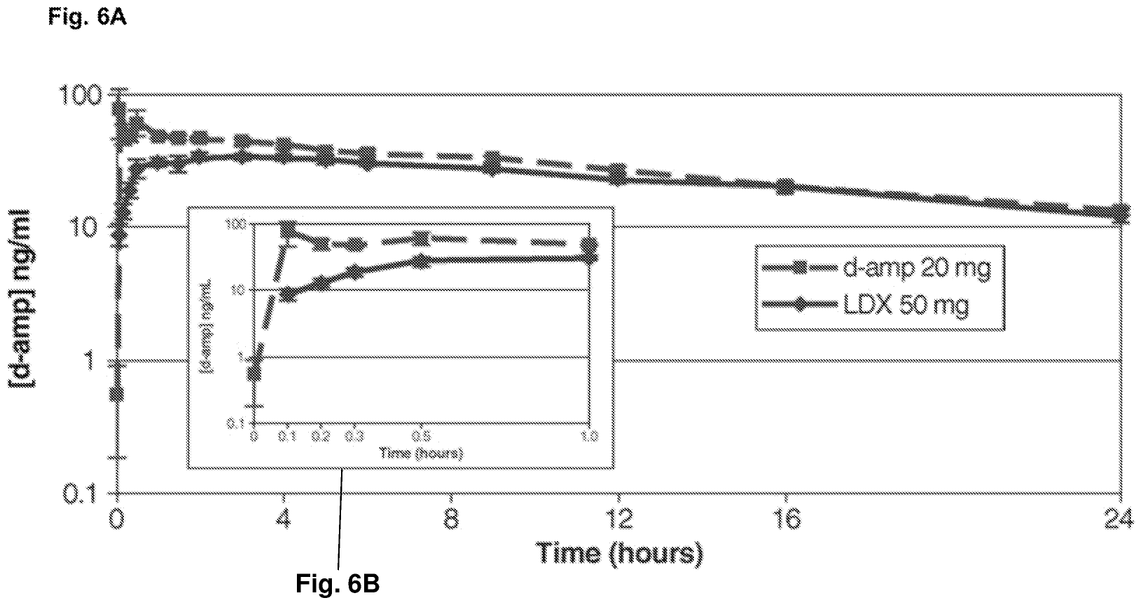

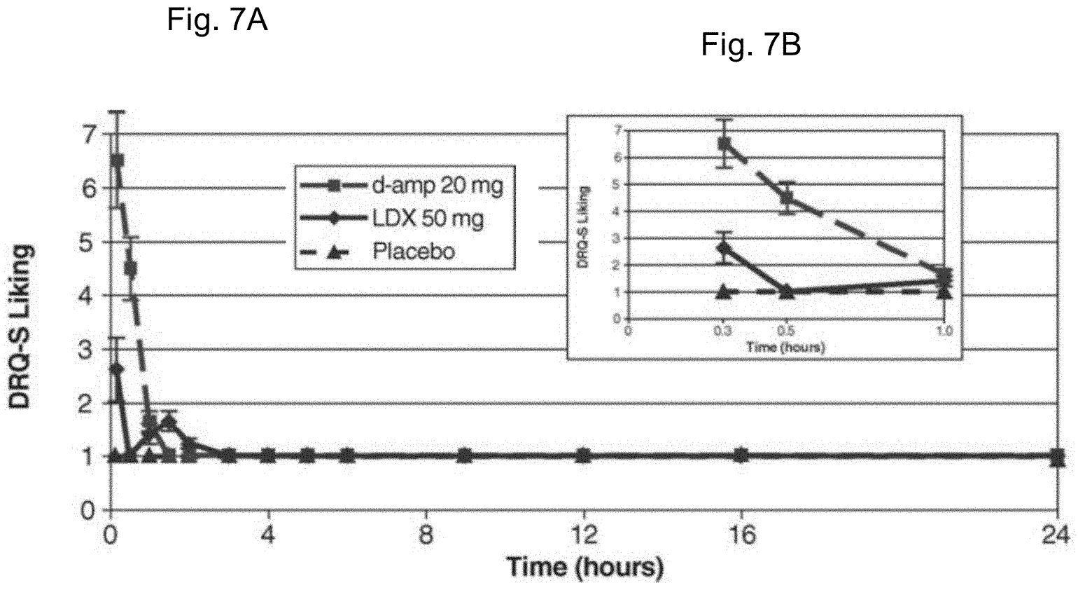

ProMDMA compounds have attenuated acute effects including reduced and slowed increases in drug liking, reduced and slowed increases in blood pressure, and reduced and slowed increases in any anxiety at effect onset. This is based on known data comparing effects of lisdexamfetamine and d-amphetamine regarding abuse-related measures such as drug liking (Jasinski & Krishnan, 2009a; Jasinski & Krishnan, 2009b) (FIGS. 6A-9).

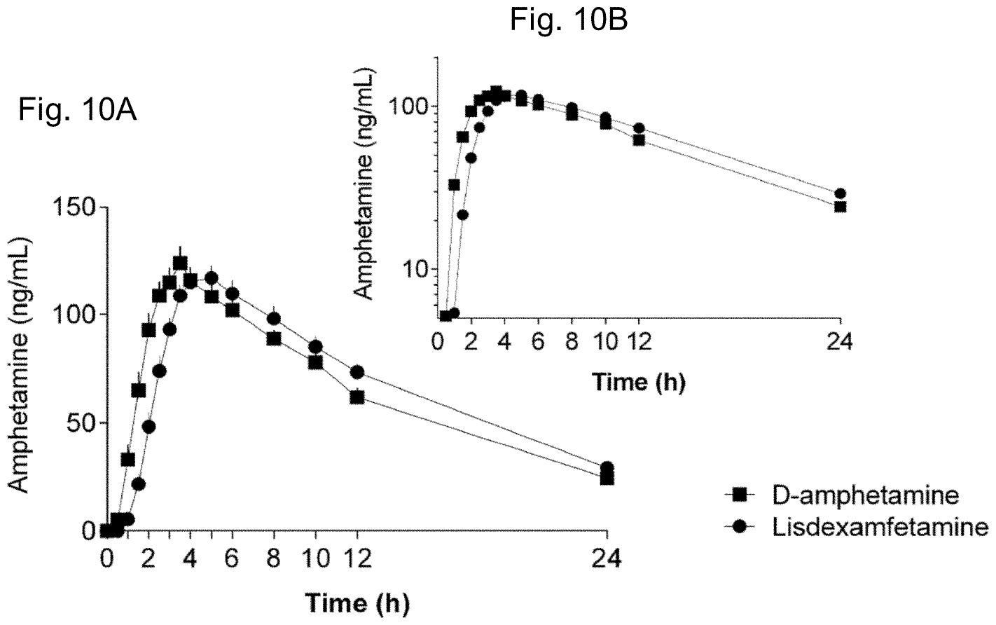

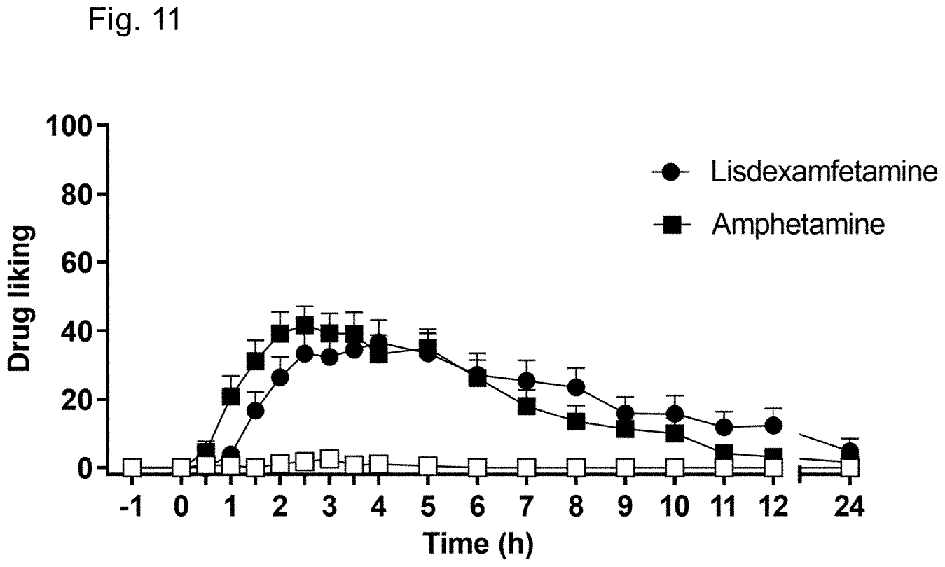

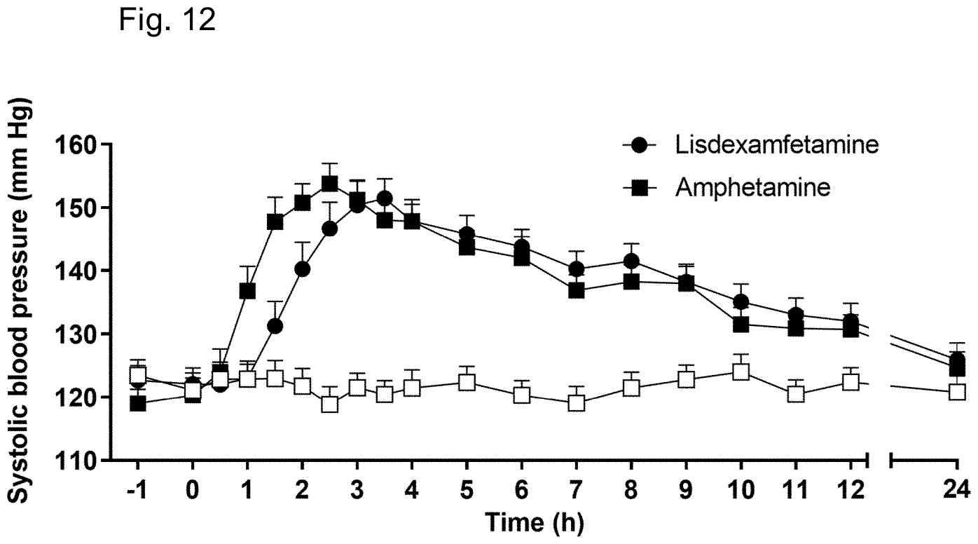

The present invention provides advantages with the prodrug concept not only regarding abuse-related effects but also with reduced anxiety ratings and reduced cardiovascular stimulation with the prodrug formulation and thus a better benefit versus adverse effect profile of the prodrug compared with the administration of the active substance. This effect is obtained by the slowed release of the active substance (MDMA) from the prodrug compound (proMDMA) producing moderated slowed increases in plasma levels of psychoactive substance (MDMA) compared to direct administration of psychoactive substance. Additionally, the published reports of reduced drug liking with orally administered lisdexamfetamine versus d-amphetamine were observed only in one study (Jasinski et al., 2009a) but not in another (Dolder et al., 2017) (FIG. 8). Unexpectedly, another very detailed and solid experimental study showed onset (10% of the individual maximal response as threshold) and peak times of the amphetamine concentration-time curve were longer after lisdexamfetamine administration compared with d-amphetamine, but no differences were found in the maximal concentrations (Dolder et al., 2017) (FIGS. 10A-10B). Additionally, the subjective drug effect-time curves including drug liking ratings were shifted to the right consistent with significantly longer time-to-effect-onset (Tₒₙₛₑₜ) and time-to-maximal-effect (Tₘₐₓ) values after lisdexamfetamine administration compared with d-amphetamine administration, consistent with the pharmacokinetics of the two drugs (Dolder et al., 2017) (FIG. 11). However, no differences in maximal effect (Eₘₐₓ) or area under the effect-time curve (AUEC) values were found between lisdexamfetamine and d-amphetamine (Dolder et al., 2017). There was a slight non-significant reduction and delay in the drug liking response after lisdexamfetamine vs. d-amphetamine (FIG. 11). Moreover, lisdexamfetamine and d-amphetamine produced similar increases in blood pressure (FIG. 12), heart rate, body temperature, and pupil size (Dolder et al., 2017). The blood pressure-time curves were shifted to the right because of significantly longer Tₒₙₛₑₜ values after lisdexamfetamine administration compared with d-amphetamine administration (Dolder et al., 2017). Thus, this contradicting data shows that there may not be a relevant difference between a prodrug and its active metabolite regarding peak effects or at least that such differences may depend on dosing. Thus, the benefits of the present invention are not obvious based on existing contradicting data (Dolder et al., 2017; Jasinski & Krishnan, 2009a; Jasinski & Krishnan, 2009b) and need to be specifically demonstrated and documented with experimental data for the prodrugs described in the present invention.

d-amphetamine and MDMA are different regarding molecular structure and metabolism. Importantly, lisdexamfetamine is converted to d-amphetamine which has a relatively long half-life of 8 hours and presence in human plasma (Dolder et al., 2017) and is metabolized to 4-hydroxyamphetamine which is an active metabolite but d-amphetamine is also eliminated unchanged and as hippuric acid conjugate in urine (Krishnan et al., 2008). In contrast, lysMDMA is converted to MDMA that is metabolized primarily at the methylenedioxy group which is not present in d-amphetamine. In particular, MDMA is mainly inactivated to 3,4-dihydroxymethamphetamine (HHMA) and then rapidly further metabolized to 4-hydroxy-3-methoxymethamphetamine (HMMA) by cytochrome P450 enzyme (CYP) 2D6 and catechol-O-methyltransferase (COMT) (de la Torre et al., 2000; Schmid et al., 2016b). This process will already take place during the formation of MDMA from lysMDMA and thus the kinetics of MDMA formation and metabolism after administration of lysMDMA are different from those of d-amphetamine formation and metabolism after administration of lisdexamfetamine and are characterized in the study described in the present invention and cannot simply be derived from past knowhow.

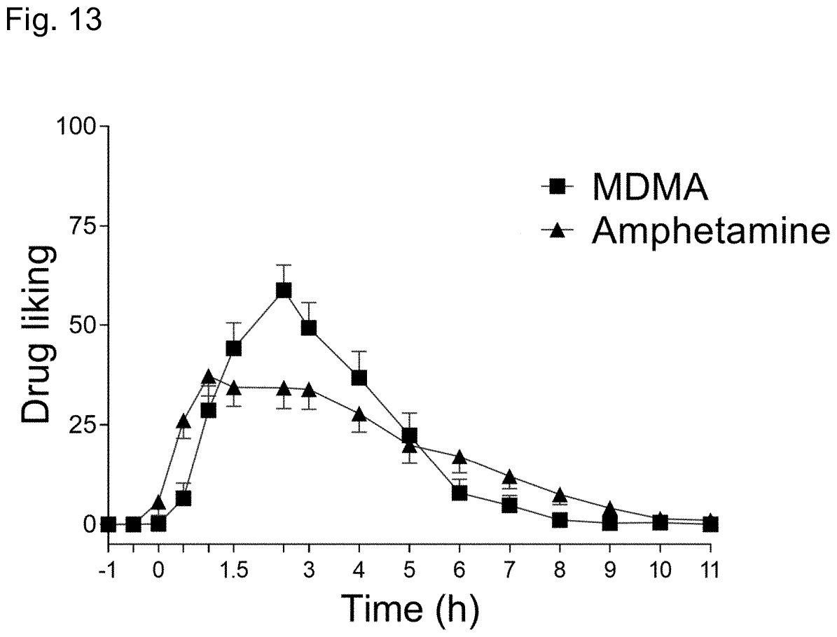

A direct comparison of the kinetics of the acute effects of d-amphetamine and MDMA also shows “slowed” kinetics for d-amphetamine compared with MDMA including lower peak effects and longer lasting subjective effects for example for ratings of liking (FIG. 13). Thus, a prodrug of MDMA will likely be different than a prodrug of d-amphetamine as there is more room for reducing Eₘₐₓ of liking and protracting the effect compared with d-amphetamine further supporting the novelty of the present innovation regarding effect modification after oral use.

Therefore, the present invention includes the design and detailed plan of an experimental study experimentally supporting the claims made.

A clinical experimental study can be performed to compare the effects of lysMDMA and lysMDA with those of MDMA and MDA, respectively, within the same participants using a randomized balanced-order (placebo-controlled) cross-over design in healthy participants. Molar equivalent doses of lysMDMA and MDMA or lysMDA and MDA are administered with a content of active drug (MDMA or MDA) corresponding to 125 mg of MDMA as the hydrochloride salt. The primary outcome measures are the plasma pharmacokinetics of MDMA and MDA, subjective drug effects including any, good, and bad drug effects as well as drug liking and anxiety; autonomic drug effects including heart rate and diastolic and systolic blood pressure. The relevant pharmacokinetic parameters regarding this invention are Cₘₐₓ, Tₘₐₓ, Tₒₙₛₑₜ, and AUC (area under the concentration-time curve). The relevant parameters regarding the effects of the substances are Eₘₐₓ, Tₘₐₓ, Tₒₙₛₑₜ and AUEC. lysMDMA/lysMDA vs MDMA/MDA will produce lower Cₘₐₓ, higher Tₘₐₓ, longer Tₒₙₛₑₜ, and similar AUC values for plasma levels of active MDMA/MDA as well as: lower Eₘₐₓ, longer Tₘₐₓ, longer Tₒₙₛₑₜ and similar AUEC levels for ratings of subjective effects and for measures of autonomic responses. This outcome would correspond to a prolonged and attenuated response to administration of lysMDMA/lysMDA as compared with MDMA/MDA. The cross-over study can include only lysMDMA and MDMA or only lysMDA and MDA or all four conditions or an additional placebo condition. The relevant comparisons regarding the present invention are lysMDMA versus MDMA and lysMDA versus MDA. The study can also include a comparison between MDMA and MDA and between lysMDMA and lysMDA to derive additional information on the difference between MDMA and MDA. Specifically, the clinical experimental data on the difference between MDMA and MDA is not available from a study validly comparing the two and such a comparison can either be integrated into the study including lysMDMA and lysMDA or can even be performed as a separate experimental study comparing only MDMA and MDA. The novel aspect of such an experimental study is presented in the following.

MDA is a psychoactive amphetamine and MDMA analog. MDA is also an active metabolite of MDMA. Peak plasma concentrations of MDA are approximately 7-10% of those of MDMA after administration of MDMA (Hysek et al., 2011; Schmid et al., 2016a). Plasma levels of MDA increase more slowly and reach a maximum later compared with levels of MDMA after administration of MDMA. Tₘₐₓ values are 2.6 and 4.7 for MDMA and MDA after administration of 125 mg MDMA to healthy subjects (Hysek et al., 2011). Additionally, the elimination half-life of MDA is 10-16 hours and longer than that of MDMA (7-10 hours) (Baggott et al., 2019; Hysek et al., 2011; Kolbrich et al., 2008). This means that effects of MDA can last longer than those of MDMA when MDA is administered as a drug. It also means that levels of the MDMA-metabolite MDA in plasma are relatively higher compared with MDMA levels towards the end of an MDMA experience and effects of MDA may contribute to some extent to the MDMA experience, in particular towards the end of the experience.

The MDMA metabolite MDA is psychoactive (Baggott et al., 2019) and has been used in the past in MDA-assisted psychotherapy similarly to MDMA (Pentney, 2001; Turek et al., 1974; Yensen et al., 1976). The pharmacology of MDA is overall relatively similar to MDMA supporting the view that MDA is an MDMA-like compound (Hysek et al., 2012; Oeri, 2020). The relative dopamine over serotonin transporter inhibition (DAT/SERT) potency ratio is a key determinant of the type of psycho-activity produced by an amphetamine compound.

Specifically, substances with a low DAT/SERT-ratio (<1) are MDMA-like empathogenic compounds while substances with a high DAT/SERT-ratio (>10) and therefore a predominant dopaminergic action are amphetamine/methamphetamine-like stimulants (Liechti, 2015; Simmler et al., 2013). For example, compounds that are MDMA-like and included in the present invention like MDMA, MBDB, MDEA and MDA have DAT/SERT ratios of 0.08, 0.09, 0.14, 0.24, respectively (Simmler et al., 2013). The benzofurans 5-APB, 6-APB have DAT/SERT ratios of 0.05 and 0.29, respectively (Rickli et al., 2015b). The aminoindane MDAI has a DAT/SERT-ratio of 0.2 (Simmler et al., 2014).

All these substances also release serotonin similar to MDMA (Rickli et al., 2015b; Simmler et al., 2013; Simmler et al., 2014). Thus, all these compounds are alike with regarding to their main action which is to release monoamines with a preference for serotonin over dopamine.

However, there are notable differences: MDA is slightly more dopaminergic than MDMA (Hysek et al., 2012; Rickli et al., 2015b). MDA also activates the 5-HT_{2A} receptor, which mediates psychedelic effects (Preller et al., 2017; Vollenweider et al., 1998b), with significantly greater potency than MDMA (Rickli et al., 2015b). Concentrations producing half-maximal effect (EC₅₀) values of 5-HT_{2A} receptor activation are 6.1 and 0.63 for MDMA and MDA, respectively (Rickli et al., 2015b). Thus, based on the pharmacological profile, MDA would be expected to exert more LSD-like psychedelic effects than MDMA.

A direct comparison of MDMA and MDA within a clinical experimental study is outstanding.

One previous study tested the effects of MDA (1.4 mg/kg orally) in 12 healthy subjects and also provided indirect comparisons with the effects of MDMA (Baggott et al., 2019). Importantly the data was obtained in different subjects and studies and is therefore not a valid comparison. The effects of MDA reportedly shared features with MDMA as well as with classical psychedelics (Baggott et al., 2019) in line with the in vitro pharmacological profile (Rickli et al., 2015b). MDA self-reported effects lasted longer than those of MDMA and up to 8 hours while MDMA effects resolved by 6 hours. MDA also produced greater perceptual changes than MDMA on the 5-Dimensions of Altered States of Consciousness Scale (Baggott et al., 2019) indicating more psychedelic-like properties.

Based on these previous data, a difference exists between MDMA and MDA and namely more psychedelic-like and longer lasting effects of MDA compared with MDMA.

Additionally, the use of lysMDA can further prolong and attenuate the MDA response and create an experience distinct from that of MDA and MDMA and desired in some patient populations. Specifically, lysMDA is useful in situations where a longer and more mixed empathogenic-psychedelic response is desired compared to the shorter and more empathogenic response to MDMA.

Other compounds with an MDA-like structure or their prodrug compositions can be used as described for MDMA or MDA within the present invention. Specifically, MDA-like compounds include MBDB, BDB, and fluorine-containing analogs of MDMA such as 2F-MDA, 5F-MDA, 6F-MDA. BDB and the fluorinated MDA compounds release 5-HT and exhibit DAT/SERT-inhibition ratios between 0.1 and 1 and are therefore similar to MDMA regarding their main pharmacological property to stimulate the serotonin over dopamine system (data on file).

The present invention provides generally for a method of treating an individual, by administering proMDMA or a proMDMA-like compound to the individual, metabolizing the prodrug, and releasing the MDMA or MDMA-like substance in the individual. This method can provide a way around or avoid metabolism in the GI tract of MDMA for metabolism elsewhere in the body, such as the liver or circulation. There are many beneficial effects of administering proMDMA or a proMDMA-like compound as opposed to the psychoactive substance without the prodrug described below.

The compositions described herein can be used in any type of substance-assisted psychotherapy similar to the intended use of MDMA or LSD or psilocybin (Danforth et al., 2018; Luoma et al., 2020; Mithoefer et al., 2016; Mithoefer et al., 2018; Trope et al., 2019).

Specifically, the compounds can be used in compound-assisted therapy for medical disorders including post-traumatic stress disorder, social anxiety, autism spectrum disorder, substance use disorder, depression, anxiety disorder, anxiety with life-threatening disease, personality disorder including narcistic or antisocial personality disorder, obsessive compulsive disorder, couple therapy, enhancement of any psychotherapy by inducing feelings of well-being connectivity, trust, love, empathy, openness, and pro-sociality, and enhancing therapeutic bond in any psychotherapy of patients or neurotic/healthy subjects.

In comparison with the use of MDMA or related psychoactive substances, the prodrug compounds described herein have a slower onset of action due to retarded kinetic properties, have longer duration of action. have reduced peak effects and thereby an attenuated effect profile, produce lower apprehension anxiety at the onset of the subjective drug effect, produce lower apprehension anxiety at the onset of the subjective drug effect, produce a slower increase in drug-liking rating scores over their acute effects, have a reduced risk of abuse and dependence, have a delayed and attenuated effect when used parenterally and thereby are abuse deterrent, and have a delayed and attenuated cardio-stimulant effect and therefore are safer to use in patients with cardiovascular disease and risk factors. Combinations of these effects can also be present.

The present invention also provides for a method of reducing anxiety while administering MDMA, by providing a slow release of MDMA or an MDMA-like substance and thereby reducing anxiety in the individual at the onset of administration. The slow release can be provided with the proMDMA or proMDMA-like substance since the pro-compound is enzymatically split into the amino acid and the psychoactive substance within the body by peptidases mainly in the circulation and release the psychoactive substance at a slowed rate compared to levels achieved by absorption rates of the psychoactive substance administered in its direct active form.

The present invention provides for a method of personalized medicine, by evaluating an individual who is in need of MDMA treatment and determining if there are characteristics of the individual present that would not be suitable for MDMA treatment and administering proMDMA or a proMDMA-like substance to the individual. For example, if the individual has cardiac issues, it would be better to treat them with proMDMA instead of MDMA. Also, if the individual had experienced anxiety at treatment onset with regular MDMA, treatment with proMDMA would be advised. A further example is indicated if a subject suffers from high levels of administered MDMA due to poor metabolism conditions: proMDMA can address and/or prevent altogether the onset effects. An even further indication can be considered if a subject has any type of gastrointestinal disorder expected to impair MDMA absorption. Hence, proMDMA, which is absorbed likely more easily and may be more suitable, is resulting in better controlled availability of MDMA in the body. This approach provides maximum efficiency and minimizes toxicity to the individual.

The present invention provides for a method of reducing abuse of MDMA, by administering proMDMA or a proMDMA-like compound and providing a delayed and attenuated effect of MDMA or a proMDMA-like compound, thereby reducing abuse. The use of a prodrug can provide, but is not limited to, reduced and slowed increases in drug liking, reduced and slowed increases in blood pressure, and reduced and slowed increases in any anxiety at effect onset because there is a delayed onset of the drug.

In comparison with MDMA, any of the other psychoactive compounds described herein and namely MDA have unique effect profiles partly distinct from MDMA making them useful alternatives to MDMA in substance-assisted therapy.

Namely, MDA can show an effect profile different from MDMA and including a longer time of action and more psychedelic effects than MDMA and desirable in selected patients. Such a distinct effect profile of MDA versus MDMA is predicted based on in vitro data and preliminary experimental data.

The compound of the present invention is administered and dosed in accordance with good medical practice, considering the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to more rapid recovery, or improvement or elimination of symptoms and other indicators as are selected as appropriate measures by those skilled in the art.

In the method of the present invention, the compound of the present invention can be administered in various ways. It should be noted that it can be administered as the compound and can be administered alone or as an active ingredient in combination with pharmaceutically acceptable carriers, diluents, adjuvants and vehicles. The compounds can be administered orally, subcutaneously or parenterally including intravenous, intramuscular, and intranasal administration. Implants of the compounds are also useful. The patient being treated is a warm-blooded animal and, in particular, mammals including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

The doses can be single doses or multiple doses over a period of several days, weeks or months. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible considering the above teachings. It is, therefore, to be understood that within the scope of the appended claims, the invention can be practiced otherwise than as specifically described.

### REFERENCES

1. Baggott M J, Garrison K J, Coyle J R, Galloway G P, Barnes A J, Huestis M A, & Mendelson J E (2019). Effects of the Psychedelic Amphetamine MDA (3,4-Methylenedioxyamphetamine) in Healthy Volunteers. J Psychoactive Drugs 51: 108-117.
2. Busto U, & Sellers E M (1986). Pharmacokinetic determinants of drug abuse and dependence. A conceptual perspective. Clin Pharmacokinet 11: 144-153.
3. Cole J C, & Sumnall H R (2003). The pre-clinical behavioural pharmacology of 3,4-methylenedioxymethamphetamine (MDMA). Neurosci Biobehav Rev 27: 199-217.
4. Creehan K M, Vandewater S A, & Taffe M A (2015). Intravenous self-administration of mephedrone, methylone and MDMA in female rats. Neuropharmacology 92C: 90-97.
5. Danforth A Exploring MDMA-assisted therapy as a new pathway to social adaptabilty for autistic adults.
6. Danforth A L, Grob C S, Struble C, Feduccia A A, Walker N, Jerome L, Yazar-Klosinski B, & Emerson A (2018). Reduction in social anxiety after MDMA-assisted psychotherapy with autistic adults: a randomized, double-blind, placebo-controlled pilot study. Psychopharmacology 235: 3137-3148.
7. Danforth A L, Struble C M, Yazar-Klosinski B, & Grob C S (2016). MDMA-assisted therapy: A new treatment model for social anxiety in autistic adults. Prog Neuropsychopharmacol Biol Psychiatry 64: 237-249.
8. de la Torre R, Farre M, Roset P N, Lopez C H, Mas M, Ortuno J, Menoyo E, Pizarro N, Segura J, & Cami J (2000). Pharmacology of MDMA in humans. Ann N Y Acad Sci 914: 225-237.
9. Dolder P C, Strajhar P, Vizeli P, Hammann F, Odermatt A, & Liechti M E (2017). Pharmacokinetics and pharmacodynamics of lisdexamfetamine compared with D-amphetamine in healthy subjects. Front Pharmacol 8: 617.
10. Hutson P H, Pennick M, & Secker R (2014). Preclinical pharmacokinetics, pharmacology and toxicology of lisdexamfetamine: a novel d-amphetamine pro-drug. Neuropharmacology 87: 41-50.
11. Hysek C M, Schmid Y, Simmler L D, Domes G, Heinrichs M, Eisenegger C, Preller K H, Quednow B B, & Liechti M E (2014). MDMA enhances emotional empathy and prosocial behavior. Soc Cogn Affect Neurosci 9: 1645-1652.
12. Hysek C M, Simmler L D, Ineichen M, Grouzmann E, Hoener M C, Brenneisen R, Huwyler J, & Liechti M E (2011). The norepinephrine transporter inhibitor reboxetine reduces stimulant effects of MDMA (“ecstasy”) in humans. Clinical pharmacology and therapeutics 90: 246-255.
13. Hysek C M, Simmler L D, Nicola V, Vischer N, Donzelli M, Krahenbuhl S, Grouzmann E, Hoener M C, & Liechti M E (2012). Duloxetine inhibits effects of MDMA (“ecstasy”) in vitro and in humans in a randomized placebo-controlled laboratory study. PLoS One 7: e36476.
14. Jasinski D R (2000). An evaluation of the abuse potential of modafinil using methylphenidate as a reference. J Psychopharmacol 14: 53-60.
15. Jasinski D R, & Krishnan S (2009a). Abuse liability and safety of oral lisdexamfetamine dimesylate in individuals with a history of stimulant abuse. J Psychopharmacol 23: 419-427.
16. Jasinski D R, & Krishnan S (2009b). Human pharmacology of intravenous lisdexamfetamine dimesylate: abuse liability in adult stimulant abusers. J Psychopharmacol 23: 410-418.
17. Kehr J, Ichinose F, Yoshitake S, Goiny M, Sievertsson T, Nyberg F, & Yoshitake T (2011). Mephedrone, compared to MDMA (ecstasy) and amphetamine, rapidly increases both dopamine and serotonin levels in nucleus accumbens of awake rats. Br J Pharmacol 164: 1949-1958.
18. Kolbrich E A, Goodwin R S, Gorelick D A, Hayes R J, Stein E A, & Huestis M A (2008). Plasma pharmacokinetics of 3,4-methylenedioxymethamphetamine after controlled oral administration to young adults. Therapeutic drug monitoring 30: 320-332.
19. Krishnan S M, Pennick M, & Stark J G (2008). Metabolism, distribution and elimination of lisdexamfetamine dimesylate: open-label, single-centre, phase I study in healthy adult volunteers. Clinical drug investigation 28: 745-755.
20. Liechti M (2015). Novel psychoactive substances (designer drugs): overview and pharmacology of modulators of monoamine signaling. Swiss Med Wkly 145: w14043.
21. Liechti M E (2014). Novel psychoactive substances (designer drugs): overview and pharmacology of modulators of monoamine signalling. Swiss Med Weekly 144: w14043.
22. Liechti M E, Gamma A, & Vollenweider F X (2001). Gender differences in the subjective effects of MDMA. Psychopharmacology 154: 161-168.
23. Luethi D, Kolaczynska K E, Walter M, Suzuki M, Rice K C, Blough B E, Hoener M C, Baumann M H, & Liechti M E (2019). Metabolites of the ring-substituted stimulants MDMA, methylone and MDPV differentially affect human monoaminergic systems. J Psychopharmacol 33: 831-841.
24. Luoma J B, Chwyl C, Bathje G J, Davis A K, & Lancelotta R (2020). A Meta-Analysis of Placebo-Controlled Trials of Psychedelic-Assisted Therapy. J Psychoactive Drugs: 1-11.
25. Mithoefer M C, Feduccia A A, Jerome L, Mithoefer A, Wagner M, Walsh Z, Hamilton S, Yazar-Klosinski B, Emerson A, & Doblin R (2019). MDMA-assisted psychotherapy for treatment of PTSD: study design and rationale for phase 3 trials based on pooled analysis of six phase 2 randomized controlled trials. Psychopharmacology.
26. Mithoefer M C, Grob C S, & Brewerton T D (2016). Novel psychopharmacological therapies for psychiatric disorders: psilocybin and MDMA. Lancet Psychiatry 3: 481-488.
27. Mithoefer M C, Mithoefer A T, Feduccia A A, Jerome L, Wagner M, Wymer J, Holland J, Hamilton S, Yazar-Klosinski B, Emerson A, & Doblin R (2018). 3,4-methylenedioxymethamphetamine (MDMA)-assisted psychotherapy for post-traumatic stress disorder in military veterans, firefighters, and police officers: a randomised, double-blind, dose-response, phase 2 clinical trial. Lancet Psychiatry 5: 486-497.
28. Mithoefer M C, Wagner M T, Mithoefer A T, Jerome I, & Doblin R (2010). The safety and efficacy of ±3,4-methylenedioxymethamphetamine-assisted psychotherapy in subjects with chronic, treatment-resistant posttraumatic stress disorder: the first randomized controlled pilot study. J Psychopharmacol 25: 439-452.
29. Mumford G K, Evans S M, Fleishaker J C, & Griffiths R R (1995). Alprazolam absorption kinetics affects abuse liability. Clinical pharmacology and therapeutics 57: 356-365.
30. Oehen P, Traber R, Widmer V, & Schnyder U (2013). A randomized, controlled pilot study of MDMA (±3,4-methylenedioxymethamphetamine)-assisted psychotherapy for treatment of resistant, chronic post-traumatic stress disorder (PTSD). J Psychopharmacol 27: 40-52.
31. Oeri H E (2020). Beyond ecstasy: Alternative entactogens to 3,4-methylenedioxymethamphetamine with potential applications in psychotherapy. J Psychopharmacol: 269881120920420.
32. Pentney A R (2001). An exploration of the history and controversies surrounding MDMA and MDA. J Psychoactive Drugs 33: 213-221.
33. Preller K H, Herdener M, Pokorny T, Planzer A, Kraehenmann R, Stampfli P, Liechti M E, Seifritz E, & Vollenweider F X (2017). The fabric of meaning and subjective effects in LSD-induced states depend on serotonin 2A receptor activation Curr Biol 27: 451-457.
34. Rickli A, Hoener M C, & Liechti M E (2015a). Monoamine transporter and receptor interaction profiles of novel psychoactive substances: para-halogenated amphetamines and pyrovalerone cathinones. European neuropsychopharmacology : the journal of the European College of Neuropsychopharmacology 25: 365-376.
35. Rickli A, Kopf S, Hoener M C, & Liechti M E (2015b). Pharmacological profile of novel psychoactive benzofurans. Br J Pharmacol 172: 3412-3425.
36. Schmid Y, Hysek C M, Simmler L D, Crockett M J, Quednow B B, & Liechti M E (2014). Differential effects of MDMA and methylphenidate on social cognition. J Psychopharmacol 28: 847-856.
37. Schmid Y, Vizeli P, Hysek C M, Prestin K, Meyer Zu Schwabedissen H E, & Liechti M E (2016a). CYP2D6 function moderates the pharmacokinetics and pharmacodynamics of 3,4-methylene-dioxymethamphetamine in a controlled study in healthy individuals. Pharmacogenet Genomics 26: 397-401.
38. Schmid Y, Vizeli P, Hysek C M, Prestin K, Meyer zu Schwabedissen H E, & Liechti M E (2016b). CYP2D6 function moderates the pharmacokinetics and pharmacodynamics of MDMA in a controlled study in healthy subjects. Pharmacogenet Genom 26: 397-401.
39. Simmler L, Buser T, Donzelli M, Schramm Y, Dieu L H, Huwyler J, Chaboz S, Hoener M, & Liechti M E (2013). Pharmacological characterization of designer cathinones in vitro. Br J Pharmacol 168: 458-470.
40. Simmler L D, Rickli A, Schramm Y, Hoener M C, & Liechti M E (2014). Pharmacological profiles of aminoindanes, piperazines, and pipradrol derivatives. Biochem Pharmacol 88: 237-244.
41. Suyama J A, Sakloth F, Kolanos R, Glennon R A, Lazenka M F, Negus S S, & Banks M L (2016). Abuse-Related Neurochemical Effects of Para-Substituted Methcathinone Analogs in Rats: Microdialysis Studies of Nucleus Accumbens Dopamine and Serotonin. J Pharmacol Exp Ther 356: 182-190.
42. Trope A, Anderson B T, Hooker A R, Glick G, Stauffer C, & Woolley J D (2019). Psychedelic-Assisted Group Therapy: A Systematic Review. J Psychoactive Drugs 51: 174-188.
43. Turek I S, Soskin R A, & Kurland A A (1974). Methylenedioxyamphetamine (MDA)-subjective effects. J Psychoactive Drugs 6: 7-14.
44. Vizeli P, & Liechti M E (2017). Safety pharmacology of acute MDMA administration in healthy subjects. J Psychopharmacol 31: 576-588.
45. Vollenweider F X, Gamma A, Liechti M E, & Huber T (1998a). Psychological and cardiovascular effects and short-term sequelae of MDMA (“ecstasy”) in MDMA-naive healthy volunteers. Neuropsychopharmacology 19: 241-251.
46. Vollenweider F X, Vollenweider-Scherpenhuyzen M F, Babler A, Vogel H, & Hell D (1998b). Psilocybin induces schizophrenia-like psychosis in humans via a serotonin-2 agonist action. Neuroreport 9: 3897-3902.
47. Yensen R, Di Leo F B, Rhead J C, Richards W A, Soskin R A, Turek B, & Kurland A A (1976). MDA-assisted psychotherapy with neurotic outpatients: a pilot study. J Nery Ment Dis 163: 233-245.

What is claimed is:

1. A compound comprising a prodrug including a psychoactive base substance attached to an amino acid.

2. The compound of claim 1, wherein said psychoactive base substance is MDMA or an MDMA-like substance.

3. The compound of claim 2, wherein the MDMA or MDMA-like substance is chosen from the group consisting of MDA, MDEA, MBDB, BDB, MDB, 2F-MDA, 5F-MDA, 6F-MDA, ethylone, MDAI, 5-IAI, 4-APB, 5-APB, 6-APB, 5-MAPDB, 6-MAPB, mixed dopaminergic-serotonergic amphetamine and their N-alkylated analogs, and active metabolites thereof.

4. The compound of claim 1, wherein said amino acid is chosen from the group consisting of lysine, alanine, arginine, asparagine, aspartic acid, cysteine, glutamine, glutamic acid, glycine, histidine, isoleucine, leucine, methionine, phenylalanine, proline, serine, threonine, tryptophan, tyrosine, and valine.

5. The compound of claim 1, wherein the amino acid is chosen from the group consisting of natural or synthetic.

6. A method of treating an individual, including the steps of:
administering a proMDMA or proMDMA-like compound to the individual;
metabolizing the prodrug in the proMDMA or proMDMA-like compound; and
releasing the MDMA or MDMA-like substance in the individual.

7. The method of claim 6, wherein the treatment is substance-assisted psychotherapy and the proMDMA or proMDMA-like compound produces at least one favorable effect compared to administering an active MDMA or MDMA-like substance alone.

8. The method of claim 7, wherein the favorable effect is chosen from the group consisting of producing less anxiety at onset of subjective effects, producing slower or smaller increases in cardiovascular activation, producing slower or smaller increases in drug liking, producing longer effects, producing more psychedelic effects, reduced abuse liability, and combinations thereof.

9. The method of claim 6, wherein the individual is treated for a medical condition chosen from the group consisting of post-traumatic stress disorder, social anxiety, autism spectrum disorder, substance-use disorder, depression, anxiety disorder, anxiety with life-threatening disease, personality disorder including narcistic or antisocial personality disorder, obsessive compulsive disorder, couple therapy, and combinations thereof.

10. The method of claim 6, further including the step of inducing feelings chosen from the group consisting of well-being, connectivity, trust, love, empathy, pro-sociality, and combinations thereof.

11. The method of claim 6, further including the step of enhancing therapeutic bonds with patients and neurotic/healthy subjects.

12. The method of claim 6, wherein said metabolizing step further includes the step of avoiding metabolism of MDMA or the MDMA-like substance in the GI tract of the individual.

13. The method of claim 6, wherein the proMDMA or proMDMA-like compound includes a MDMA or MDMA-like substance chosen from the group consisting of MDA, MDEA, MBDB, BDB, MDB, 2F-MDA, 5F-MDA, 6F-MDA, ethylone, MDAI, 5-IAI, 4-APB, 5-APB, 6-APB, 5-MAPDB, 6-MAPB, mixed dopaminergic-serotonergic amphetamine and their N-alkylated analogs, and active metabolites thereof.

14. The method of claim 6, wherein the proMDMA or proMDMA-like compound includes an amino acid chosen from the group consisting of lysine, alanine, arginine, asparagine, aspartic acid, cysteine, glutamine, glutamic acid, glycine, histidine, isoleucine, leucine, methionine, phenylalanine, proline, serine, threonine, tryptophan, tyrosine, and valine.

15. The method of claim 13, wherein the amino acid is chosen from the group consisting of natural or synthetic.

16. A method of reducing anxiety while administering MDMA, including the steps of:
providing a slow release of MDMA or an MDMA-like substance and thereby reducing anxiety in the individual at the onset of administration.

17. The method of claim 16, wherein the MDMA or MDMA-like substance is chosen from the group consisting of MDA, MDEA, MBDB, BDB, MDB, 2F-MDA, 5F-MDA, 6F-MDA, ethylone, MDAI, 5-IAI, 4-APB, 5-APB, 6-APB, 5-MAPDB, 6-MAPB, mixed dopaminergic-serotonergic amphetamine and their N-alkylated analogs, and active metabolites thereof.

18. The method of claim 16, wherein the MDMA or MDMA-like compound is attached to an amino acid chosen from the group consisting of lysine, alanine, arginine, asparagine, aspartic acid, cysteine, glutamine, glutamic acid, glycine, histidine, isoleucine, leucine, methionine, phenylalanine, proline, serine, threonine, tryptophan, tyrosine, and valine.

19. The method of claim 18, wherein the amino acid is chosen from the group consisting of natural or synthetic.

20. A method of personalized medicine, including the steps of:
evaluating an individual who is in need of MDMA treatment and determining if there are characteristics of the individual present that would not be suitable for MDMA treatment; and
administering proMDMA or a proMDMA-like compound to the individual.

21. The method of claim 20, wherein the individual has a condition chosen from the group consisting of cardiac issues, anxiety experienced at treatment onset with regular MDMA, high levels of administered MDMA due to poor metabolism conditions, and gastrointestinal disorders that impair MDMA absorption.

22. The method of claim 20, wherein the proMDMA or proMDMA-like compound includes a MDMA or MDMA-like substance chosen from the group consisting of MDA, MDEA, MBDB, BDB, MDB, 2F-MDA, 5F-MDA, 6F-MDA, ethylone, MDAI, 5-IAI, 4-APB, 5-APB, 6-APB, 5-MAPDB, 6-MAPB, mixed dopaminergic-serotonergic amphetamine and their N-alkylated analogs, and active metabolites thereof.

23. The method of claim 20, wherein the proMDMA or proMDMA-like compound includes an amino acid chosen from the group consisting of lysine, alanine, arginine, asparagine, aspartic acid, cysteine, glutamine, glutamic acid, glycine, histidine, isoleucine, leucine, methionine, phenylalanine, proline, serine, threonine, tryptophan, tyrosine, and valine.

24. The method of claim 23, wherein the amino acid is chosen from the group consisting of natural or synthetic.

25. A method of reducing abuse of MDMA, including the steps of:
administering proMDMA or a proMDMA-like compound; and
providing a delayed and attenuated effect of MDMA or a MDMA-like substance, thereby reducing abuse.

26. The method of claim 25, wherein the proMDMA or proMDMA-like compound has a low bioavailability in parenteral routes.

27. The method of claim 25, wherein said providing step is further defined as providing an effect chosen from the group consisting of reduced and slowed increases in drug liking, reduced and slowed increases in blood pressure, and reduced and slowed increases in any anxiety at effect onset.

28. The method of claim 25, wherein the proMDMA or proMDMA-like compound includes a MDMA or MDMA-like substance chosen from the group consisting of MDA, MDEA, MBDB, BDB, MDB, 2F-MDA, 5F-MDA, 6F-MDA, ethylone, MDAI, 5-IAI, 4-APB, 5-APB, 6-APB, 5-MAPDB, 6-MAPB, mixed dopaminergic-serotonergic amphetamine and their N-alkylated analogs, and active metabolites thereof.

29. The method of claim 25, wherein the proMDMA or proMDMA-like compound includes an amino acid chosen from the group consisting of lysine, alanine, arginine, asparagine, aspartic acid, cysteine, glutamine, glutamic acid, glycine, histidine, isoleucine, leucine, methionine, phenylalanine, proline, serine, threonine, tryptophan, tyrosine, and valine.

30. The method of claim 29, wherein the amino acid is chosen from the group consisting of natural or synthetic.
